(19) United States
(12) Patent Application Publication
(10) Pub. No.: US 2023/0324386 A1
(43) Pub. Date: Oct. 12, 2023

(54) Novel Coronavirus Testing

(71) Applicant: Olisa Care S.à r.l., Luxembourg (LU)

(72) Inventors: Jixian LAN, Shanghai (CN); Guanghua YANG, Shanghai (CN); Qiang ZHUANG, Shanghai (CN)

(21) Appl. No.: 18/007,764

(22) PCT Filed: Jun. 2, 2021
(86) PCT No.: PCT/EP2021/064797

§ 371 Date: Dec. 2, 2022

(30) Foreign Application Priority Data
Jun. 2, 2020 (LU) 101824

(51) Int. Cl. G01N 33/569; G01N 33/543; C07K 14/165

(52) U.S. Cl. CPC G01N 33/56983; C07K 14/165; G01N 33/54388; G01N 2333/165; G01N 2469/20

(57) ABSTRACT

The invention proposes a testing device and method for detecting infection with or immunity to SARS-Cov-2 of a subject, the method comprising the following steps: (i) contacting a urine sample from the subject with an application area of a testing device comprising a urine sorption material defining a sequence of said application area, a conjugate area and a testing area, the areas being in direct or indirect capillary flow communication with each other, (ii) allowing the urine sample to flow by capillarity from said application area to said conjugate area, said conjugate area comprising a testing conjugate movably held therein, wherein said testing conjugate comprises or consists of a polypeptide having an amino acid sequence that shares sequence identity of at least 90%, preferably at least 95%, with a SARS-CoV-2 protein or fragment thereof, coupled to a first colored marker, (iii) allowing the urine sample to continue to flow by capillarity to the testing area, said testing area comprising a testing sub-area with immobilized anti-human IgA antibodies, and (iv) determining infection with or immunity SARS-CoV-2 of the subject by visually inspecting the testing sub-area of the testing area for a color build-up, wherein the presence of a color build-up is indicative of said infection with or immunity to SARS-CoV-2.

### SEQUENCE LISTING

This application contains a sequence listing filed in electronic form as an ASCll.txt file entitled “Sequence-listings-accent-removed.txt” created on Jun. 2, 2020; and modified to US ASCII format for part of applicant name on May 24, 2023; and the size is 48 KB. The content of the sequence listing is incorporated in its entirety.

### TECHNICAL FIELD

The present invention generally relates to a novel testing device and method for an infection of a subject with or immunity of a subject to the so-called novel coronavirus, also called 2019 novel coronavirus (2019-nCoV) or SARS-CoV-2.

### BACKGROUND ART

In December of 2019, there appeared a series of pneumonia cases with unclear causes in the city of Wuhan located in Hubei Province, China. The infected had primary clinical symptoms of fever and weakness, with a main respiratory tract symptom of dry cough, along with gradually emerging dyspnoea. Those with serious illness showed acute respiratory distress syndrome, septic shock, and metabolic acidosis and coagulation disorder that were difficult to correct. A portion of sufferers had mild symptoms at onset, possibly not developing a fever. The majority of sufferers had moderate symptoms, with a good prognosis. A minority of sufferers developed grave disease conditions, even up to death. Although symptoms are similar to viral pneumonia, a kind of type of novel coronavirus was identified in the lower respiratory tract of an infected person, which was later named the 2019 novel coronavirus (2019-nCoV) or SARS-CoV-2.

Coronaviruses are a virus that affects both humans and animals, often the source of infection for these are areas of frequent contact between wild animals and humans. After many scientific studies it has been verified that the 2019-nCoV virus originated in the natural world. However up to the present moment mechanisms of infection are not yet clear, and the transmission speed, incubation period, high pathogenicity, and human to human transmission of the virus, etc., have resulted in there being a great number of infected persons in many nations all over the world, as well as an increase in mortality. Currently 2019-nCoV has become a pandemic, and has made a colossal impact on the social stability and global economy of all of humanity.

Facing a situation of the global spread of the 2019 Novel Coronavirus disease (named COVID-19), which is a serious pandemic, quick identification of those infected with novel coronavirus (2019-nCoV) is of the highest importance in preventing and controlling the pandemic. The gold standard of 2019-nCoV testing is nucleic acid testing, however in clinical work, factors including differences in specimen types, specimen quality, and patient conditions can lead to nucleic acid testing producing false negative results. Furthermore, nucleic acid testing relies on laboratory testing apparatuses, and also there exists the problem of the long time it takes to carry out testing, and the inability to make testing suitable for large-scale screening at points of care. Therefore, the question of how viral infections may be accurately and quickly identified is an important challenge. In this regard, serum specific antibodies, which are important immune response products arising from the body’s resistance to viral infection, were quickly used in laboratories to test for the 2019 novel coronavirus, and in concert with nucleic acid testing for the fast diagnosis and screening of COVID-19 patients.

The diagnostic model based on colloidal gold fast immunochromatography has increasingly received attention, where fast antibody testing can compensate for the time window and sensitivity problems of nucleic acid testing, and provide relevant information for measures to control infection. In the diagnosis of new and newly emerging human coronaviruses, antibody testing is especially important. Under these conditions, there is the possibility that positive detection of viral DNA of infected persons may not be possible, especially during the early stages of the disease, however it can be retrospectively proven that an immune response was already produced. In the identification of SARS-CoV-2, especially when quick antigen tests and/or molecular assays cannot be used and are unstable, then serology can serve as a supplemental diagnostic tool. A recent study found that 5 days after onset, IgM and IgG antibodies were found in each one of a group of 39 SARS-CoV-2 infected individuals. When collection of nasopharyngeal swabs is unsuitable and the results of molecular assays are not satisfactory, serum assays can be used to supplement diagnoses of SARS-CoV-2 infection. Quick antibody tests can make up for the sensitivity and time window problems of nucleic acid testing, and provide relevant information for measures to control infection.

Currently, the 2019-nCoV/SARS-CoV-2 antibody tests on the market are based on the testing of blood or serum samples. Generally, those tests based on venous blood or serum rely on specialized medical organizations such as hospitals, etc., and require professionals to carry out blood collection. The range of use of these testing products is therefore greatly limited by their sites of application. Moreover, those based on home use require blood from fingertips, thus requiring matching a needle for fingertip blood drawing and pipette, as well as trained healthcare staff. This invasive method is not acceptable to all people, and as a result the universalization of this testing product has definite limits. As a result, the invention of a simpler and more universal testing product for novel coronavirus was pressing.

### TECHNICAL PROBLEM

It is therefore an object of the present invention to provide a test method and a corresponding testing device for easily, rapidly and yet reliably detecting if a subject has been infected by SARS-CoV-2 and has produced antibodies or not. The test method and device should be non-invasive, should not cause pain and should be usable by any person without the assistance of medical or otherwise trained staff and without further equipment, in particular by any person without help and even without particular skills.

### GENERAL DESCRIPTION OF THE INVENTION

To achieve this object, the present invention proposes, in a first aspect, a method for detecting an infection of a subject with SARS-Cov-2 coronavirus or immunity of a subject towards SARS-Cov-2 coronavirus, the method comprising the following steps:
(i) contacting a urine sample of the subject with an application area of a testing device comprising a sorption material defining a sequence of said application area, a conjugate area and a testing area, the areas (in that order) being in direct or indirect capillary flow communication with each other,
(ii) allowing the urine sample to flow by capillarity from said application area to said conjugate area, said conjugate area comprising a testing conjugate movably held therein, wherein said testing conjugate comprises or consists of a (first) polypeptide having an amino acid sequence that shares sequence identity of at least 90%, preferably at least 95%, with a SARS-CoV-2 protein or fragment thereof, coupled to a first colored marker,
(iii) allowing the urine sample to continue to flow by capillarity to the testing area, said testing area comprising a testing sub-area with immobilized anti-human IgA antibodies, and
(iv) determining the subject’s infection with SARS-Cov-2 or immunity towards SARS-CoV-2 by visually inspecting the testing sub-area of the testing area for a color build-up, wherein the presence of a color build-up is indicative of said infection with SARS-Cov-2 or immunity towards SARS-CoV-2.

In a second aspect, the invention proposes a testing device for detecting infection of a subject with SARS-Cov-2 coronavirus or immunity of a subject to SARS-Cov-2 coronavirus, the testing device comprising a sorption material defining a sequence of an application area, a conjugate area and a testing area, the areas (in that order) being in direct or indirect capillary flow communication with each other, wherein said application area is configured for receiving a urine sample of a subject, wherein said conjugate area comprises a testing conjugate movably held therein, wherein said testing conjugate comprises or consists of a (first) polypeptide having an amino acid sequence that shares sequence identity of at least 90%, preferably at least 95%, with a SARS-CoV-2 protein or fragment thereof, coupled to a first colored marker, wherein said testing area comprises a testing sub-area with immobilized anti-human IgA antibodies, and wherein said testing sub-area is configured for visually inspecting any color build-up, and wherein a color build-up is indicative of said infection of the subject with SARS-CoV-2 or immunity of the subject toSARS-CoV-2.

In a third aspect, the invention encompasses the use of a testing conjugate comprising or consisting of a polypeptide having an amino acid sequence that shares sequence identity of at least 90%, preferably at least 95%, with a SARS-CoV-2 protein or fragment thereof, coupled to a first colored marker, for detecting infection of a subject with SARS-Cov-2 or immunity of a subject to SARS-Cov-2. In particular, the invention comprises the detection of an infection of a subject with SARS-Cov-2 or immunity of a subject to SARS-Cov-2, the detection being made based on a urine sample of a subject.

The present invention has major benefits over existing testing methods and devices for detecting if a subject has been infected with SARS-CoV-2 coronavirus, even if (still) asymptomatic. Indeed, first of all the method and testing device as proposed by the present invention are easy to implement, do not require assistance or complex equipment, are non-invasive, do not cause pain or even the fear thereof and the results is available within a few minutes. As the testing device is relatively inexpensive and the method quick and easy, a same subject can be tested/can test himself (self test) as often as deemed necessary or useful.

A direct beneficial consequence thereof is that a wide application of this testing device and method prevents the circulation of suspected infected persons and reduces the latent risk of transmission and allows one to know one’s immunity status following an infection or vaccination .

The invention is based on the findings that a subject infected by the SARS-CoV-2 coronavirus will produce among others antibodies (IgA and/or IgG) specific to said coronavirus and these IgA and IgG are excreted with the subject’s urine. The terms “antibodies specific to [said] coronavirus” or “antibodies specific to SARS-CoV-2” in this context mean any antibody, whether in monomeric or dimeric form, produced by said subject in response to infection with the SARS-CoV-2 coronavirus and variants. Indeed, the terms “SARS-CoV-2” and “2019-nCoV” in the context of the present disclosure include any mutant variants of said virus.

The invention makes thus use of SARS-CoV-2 proteins or fragments thereof as an antigen to IgA and/or IgG antibodies produced by a subject in reaction to a SARS-CoV-2 infection and excreted with the urine. These proteins or fragments can be easily, rapidly and relatively inexpensively mass produced e.g. as recombinant proteins from the corresponding recombinant DNA sequences, such as e.g. SEQ ID NO: 1 or fragments thereof. The DNA sequence may comprise an end sequence encoding one or more C-terminal tags, such as His tags, e.g. the 6 His encoding end sequence of SEQ ID NO:1.

Further beneficial variants and advantages of the present invention will become apparent from the further description and examples below.

Advantageously, the SARS-CoV-2 protein or fragment thereof has an amino acid sequence comprising that of SARS-CoV-2 surface glycoprotein (Coronavirus S protein, Coronavirus spike protein), that of SARS-CoV-2 surface glycoprotein S1(Coronavirus S1 protein), of SARS-CoV-2 surface glycoprotein S2 (Coronavirus S2 protein), of SARS-CoV-2 RBD protein, of SARS-CoV-2 Nucleocapsid phosphoprotein N (Coronavirus N protein), or of fragments of at least 150, preferably at least 180, most preferably at least 190 consecutive amino acids thereof.

In preferred embodiments, the (first) polypeptide has an amino acid sequence that shares sequence identity of at least 90%, preferably at least 95%, with SEQ ID NO:2, SEQ ID NO:3, SEQ ID NO:4, SEQ ID NO:5, SEQ ID NO:6, SEQ ID NO:7, SEQ ID NO:8, or any fragment of at least 150, preferably at least 180, most preferably at least 190 consecutive amino acids thereof. In particularly preferred embodiments, the (first) polypeptide has the amino acid sequence of SEQ ID NO:2, SEQ ID NO:3, SEQ ID NO:4, SEQ ID NO:5, SEQ ID NO:6, SEQ ID NO:7 or SEQ ID NO:8.

The first colored marker may be any appropriate marker known to the skilled person. Preferably, in the present invention, said first colored marker is selected among nanometer-sized gold particles, latex particles, ...

In still further advantageous embodiments, the method and testing device may comprise a plurality of different SARS-CoV-2 proteins or fragments thereof or different (first) polypeptides coupled to identical or different markers. Said SARS-CoV-2 proteins or fragments thereof may be two or more of those mentioned above, or at least one of those mentioned above with other SARS-CoV-2 proteins or fragments.

In particularly advantageous embodiments, the method comprises a control feature to verify if the testing device is working properly. Hence, it is preferred that the conjugate area further comprises a control conjugate movably held therein, wherein said control conjugate comprises a control polypeptide coupled to a second colored marker and said testing area comprising a control sub-area with immobilized anti-(control polypeptide) antibodies. In such instances, the method further comprises, before or after step (iv), the step of

(iv′) determining a correct sample flow within the testing device by visually inspecting the control sub-area of the testing area for a color build-up, wherein the presence of a color build-up is indicative of said correct sample flow within the testing device.

In particularly preferred embodiments of the method, said conjugate area further comprises a control conjugate movably held therein, wherein said control conjugate comprises a control polypeptide coupled to a second colored marker and said testing area comprising a control sub-area with immobilized anti-(control polypeptide) antibodies upstream or downstream of the testing sub-area, and wherein said control sub-area is configured visually inspecting any color build-up, wherein a color build-up is indicative of a correct sample flow within the testing device.

It is of particular advantage in the present invention having such a control feature that the control polypeptide is selected among immunoglobulins, preferably IgA, IgG, ..., more preferably non-human IgG, such as rabbit IgG, mouse IgG, ...

As for the first colored marker, the second marker may also be any appropriate marker known to the skilled person. Preferably, in the present invention, said second colored marker is selected among nanometer-sized or colloid gold particles, latex particles, ...

In such preferred methods and testing devices of the invention, the absence of color build-up in the control sub-area in step (iv′) is indicative that the test result of step (iv) should be disregarded and the method repeated with a fresh testing device.

The sorption material is in particular a urine sorption material, preferably selected among porous capillary lateral flow materials, such as glass fiber or cellulosic materials, preferably modified cellulosic material, such as nitrocellulose. Advantageously, the sorption material of the testing device is supported on a (non-sorptive) backing pad, preferably made of glass fiber, polyester film, a non-woven, ...

In preferred embodiments of the testing device, said conjugate area further comprises a control conjugate movably held therein, wherein said control conjugate comprises a control polypeptide coupled to a second colored marker and said testing area comprising a control sub-area with immobilized anti-(control polypeptide) antibodies upstream or downstream of the testing sub-area, and wherein said control sub-area is configured visually inspecting any color build-up, wherein a color build-up is indicative of a correct sample flow within the testing device.

As already briefly indicated above, urine may serve as a source for more universally applicable non-invasive testing of SARS-CoV-2 antibodies. Many of the antibodies in the urine are secretion type antibodies, mainly IgA and/or IgG. The testing device of this invention is thus based on the presence of antibodies in urine, and as a result testing can be carried out in regards to the specific antibodies present within the urine of individuals who are infected with SARS-CoV-2. The testing device and method of the present invention generally includes the following technological characteristics: 1) this is a non-invasive test that is simple to use, and only requires a few drops of urine, which is easy, convenient and fast. In a few minutes (e.g. 10 to 20 minutes), the results can be obtained, and this test can be used at end point residences for self-testing. 2) it has been determined that the hook effect (or prozone effect) is overcome by use of an integrated sandwich method and capture method, resulting in higher test sensitivity and significantly increased rates of detection. In early clinical testing, morning urine negative detection rates were 91% or more; 3) since the sample examined by the testing device is collected urine, there is no need for specialized equipment, and therefore one need only insert one end of the testing device into the urine. After waiting several minutes, the paper can be retrieved and the results can be seen; 4) since the application area of the testing device is inserted directly into the urine sample, there is no need to provide the testing device with plastic holding/protecting shells, making production fast and convenient, with increased efficiency. Due to this, the wide application of this testing device and method can avoid the circulation of persons who have suspected infections and can reduce the latent risk of transmission. At the same time, it is even more favorable in terms of the health monitoring of high-risk groups. Also, this invention can prevent the propagation of sources of infection by asymptomatic individuals, and is an important detection means for saving on the consumption and exhaustion of medical manpower and materials.

The terms “polypeptide” and “protein” are used interchangeably to refer to a polymer of amino acid residues, and are not limited to a minimum length. Polypeptides, including the antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site- directed mutagenesis, or may be accidental, such as through mutations of hosts which produce the proteins or errors due to PCR amplification. In particular the protein or polypeptide sequences may comprise one or more C-terminal tags, such as His tags.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the protein or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

### BRIEF DESCRIPTION OF THE DRAWINGS

Preferred embodiments of the invention will now be described, by way of example, with reference to the accompanying drawings in which:

FIG. 1 is a block schematic diagram of a confirmed production process of preferred testing devices of the present invention.

FIG. 2 is schematic view of the manipulation of a preferred testing device of the invention during and after sampling.

FIG. 3 is a schematic view of potential results of the SARS-CoV-2 testing according to preferred embodiments of the present invention.

Further details and advantages of the present invention will be apparent from the following detailed description of several not limiting embodiments with reference to the attached drawings.

### DESCRIPTION OF PREFERRED EMBODIMENTS

### 1. Technological Principles

In particularly preferred embodiments, the testing device and method takes advantage of the principle of immunochromatography, and uses the capture method to test for the presence of novel coronavirus SARS-CoV-2 (also called 2019-nCoV) antibodies in human urine samples. During testing, when a sample contains SARS-CoV-2 antibodies and has a density that is greater than or equal to a lower limit of detection, the antibodies and antigen markers are conjugated, and the conjugate is then captured again in the second antibody (anti-µ chain antibody/ anti-human IgG antibody) detection area (testing sub-area T), which forms a red reaction line, at which time the result is determined to be positive; otherwise if no line becomes visible then the result is deemed negative. Under normal circumstances, the quality control area (control sub-area C) will always become colored, in order to indicate that the test was valid.

Use: used for in vitro qualitative detection of novel coronavirus (2019-nCoV) antibodies in human urine samples.

### 2. Technological Contents

Many of the antibodies in the urine are secretion type antibodies, mainly IgA and/or IgG. Therefore, in order to increase the sensitivity of the test, the testing device advantageously uses a double antigen sandwich capture method to detect antibodies in the sample. The marker of the conjugate gold marker contains recombinant antigens of novel coronavirus, and the film of the paper test strip contains (recombinant antigens and mouse anti-human IgA antibodies). When the tested urine sample has the corresponding antibodies, the areas with the antibodies will bind with antigens on the gold marker conjugate. When the gold marker conjugate - antibody is chromatographed on the testing area of the testing strip, on the one hand the remaining antibody areas can bind to the antigens there, and on the other hand, the antibodies are bound by the antibodies on the film and are recognized. In employing the aforementioned two mechanisms, the gold marker will be fixed to the corresponding testing area, with this area displaying a red indicator. On the contrary, if the sample does not contain antibodies, or contains a density of antibodies lower than the lower limit of detection of the reagent, then the gold indicator will not be fixed to the testing area, and the area will not display a red color. The mixture on the gold marker conjugate includes a marker with rabbit IgG gold particles, and the testing paper strip control area (C line) has sheep anti-rabbit antibodies. Regardless of whether the testing area displays a color or not, this area will show a color by means of the fixed marker with rabbit IgG gold particles. If this area does not display a color, then this indicates the test is not valid.

Using the combination of the aforementioned two methods, the sensitivity of antibody testing can be increased. In terms of developing accurate testing of urine samples, this research and development led to the invention of a sample pad, where after a sample is placed on the pad and suitably adjusted to a reaction system, the sample produces very good detection.

The sample pad of the testing paper of this invention is preferably a glass fiber, polyester film or a non-woven that was treated in a solution and oven dried.

### 3. Handling of the Sample Pad Includes

1) Buffer system: Use one or several buffer liquid composites including 0.01-0.5 M, of the following listed below. Including but not limited to: tris-hydrochloric acid, boric acid- borax, phosphate buffer, hepd buffer system;
2) Macromolecules: 0.1-3% of one or several of the following macromolecules. Bovine serum protein, casein, polyethylene glycol, gelatin.
3) Salt content: 0.1% - 5% sodium chloride / magnesium chloride.
4) Colloidal protection constituents: 0.1 %-5%, PVP.
5) Preservative: 0.01%-1%, sodium azide, peoclin.
6) Ultra-pure water preparation.

After soaking in the solution, the glass fiber is placed in a 25-50° C. oven for oven drying before use.

### 4. Manufacturing Process and Response System

TABLE 1

Outline of manufacturing process

| | | |
|---|---|---|
| Process | Quality control point | Criteria |
| Coated film | The width and position of the testing line and quality control line | The distance between the quality control line and the testing line is approximately 4.5 ± 0.5 mm, the width of the line is approximately, 0.8-1.0 mm. |
| NC oven drying | Temperature in the oven | temperature 45±1° C.; humidity ≤25%; drying 12 hours |
| Manufacture of the bonding pad | amount of liquid added | Each glass fiber pad has 30 ml of liquid added to it, which is distributed evenly. |
| Drying of the bonding pads | Colloidal gold pads are evenly dried | oven 45±1° C., drying for 12 hours |
| Affixing of film | Ambient temperature and humidity for affixing of film | Temperature 18-26° C.; humidity ≤25%; |
| Cutting | Width of cut strips, ambient temperature and humidity of cutting | The width of the film strip must be 2.5 mm, the tolerance is the required strip width (2.5 mm / 3 mm etc.) ±0.05 mm; temperature 18-26° C.; humidity ≤25%; |
| Assembly | Ambient temperature and humidity | Temperature 18-26° C.; humidity ≤25%; |

### 5. Reaction System

### 1) Sample Collection and Processing

Collect urine from first urination in the morning, clean mid-morning urine. No processing needed, may be tested directly.

### 2) Sample Requirements

Females: Before sample collection use soap or 0.1% liquor potassii permanganatis solution to flush the vulva, use fingers to open the labia, and on urination discard first portion of urination but do not stop urination, and collect 10-20 ml of urination from the middle period and store in a sterile receptacle.

Males: Before sample collection use soap or 0.05% - 0.1% of povidone iodine (iodine) solution to disinfect the urethra, after wiping clean, replace foreskin, and on urination discard first portion of urination but do not stop urination, and collect 10-20 ml of urination from the middle period and store in a sterile receptacle.

### 3) Amount of Sample

Directly insert the reagent into the urine sample, to ensure the chromatographic amount is sufficient.

### 6. Reactive Mode

This reagent kit utilizes the principle of immunochromatography, and uses the capture method to test for the presence of novel coronavirus (2019-nCoV) antibodies in human urine samples. During testing, when a sample contains novel coronavirus (2019-nCoV) antibodies and has a density that is greater than or equal to the lower limit of detection, the antibodies and antigen markers are conjugated, and the conjugate is then captured again in the second antibody (anti-µ chain body/ anti-human IgG antibody) detection area (T), which forms a red reaction line, at which time the result is determined to be negative; otherwise if no line becomes visible then the result is deemed positive. Under normal circumstances, the quality control area (C) will always become colored, in order to indicate that the test was valid.

### 7. Flowchart of the Manufacturing Process

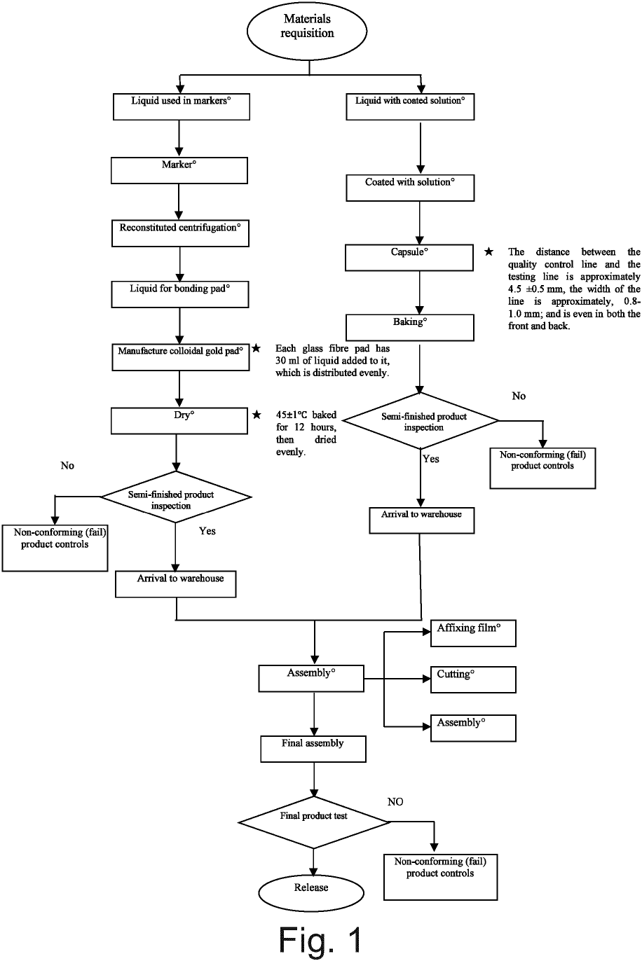

Performance evaluation was carried out on three lots of products from a test production run. The quality of these products all satisfied the reagent kit performance requirements, and did not have any obvious discrepancies. A confirmed production process flowchart is as pictured in FIG. 1. The highlighted working procedures (° marking) are carried out in a 100,000 class clean area, and the rest are carried out in a common production area. * Marking is for key control points of the working process.

### 8. Example of Package Description

### [Product Name]

Common name: Urine Novel Coronavirus (2019-nCoV) Antibody Reagent Kit (Colloidal Gold)

### [Packaging Specifications]

Type: doses for: 1 person/sachet, 5 people/box, 10 people/box, 20 people/box, 25 people/box, 50 people/box.

### [Expected Usage]

This reagent kit is used for in vitro qualitative detection of novel coronavirus (2019-nCoV) antibodies in human urine samples.

If the result of the test is positive then subsequent confirmation is still required, and if the test result is negative the possibility of infection cannot be discarded. After an interval of 5 days, a negative sample can be tested a second time.

The development of laboratory testing for novel coronavirus must comply with the requirements of the “Technical guide on laboratory testing for novel coronavirus infection pneumonia,” etc., and carry out biosafety work correctly.

### [Testing Principles]

This reagent kit utilizes the principle of immunochromatography, and uses the capture method to test for the presence of novel coronavirus (2019-nCoV) antibodies in human urine samples. During testing, when a sample contains novel coronavirus (2019-nCoV) antibodies and has a density that is greater than or equal to the lower limit of detection, the antibodies and antigen markers are conjugated, and the conjugate is then captured again in the second antibody (anti-µ chain body/ anti-human IgG antibody) detection area (T), which forms a red reaction line, at which time the result is determined to be negative; otherwise if no line becomes visible then the result is deemed positive. Under normal circumstances, the quality control area (C) will always become colored, in order to indicate that the test was valid.

### [Primary Components]

Testing strip, desiccant, urine cup.

Testing strip: the testing strip is composed of a nitrocellulose (NC) film, sampling pad, conjugation pad, water absorption paper, and PVC board. The nitrocellulose film includes anti-µ chain body/anti-human IgG antibody, anti-rabbit polyclonal antibody, and the conjugation pad includes 2019-nCoV recombinant antigens, and rabbit IgG.

### [Storage Conditions and Valid Period]

Store at 2° C.-30° C., valid for 12 months.

After opening the tinfoil bag, use the testing strip within 30 minutes.

Date of production: see product label.

Expiration date: see product label.

### [Specimen Requirements]

Urine sample (morning urine is best).

Testing should be carried out on samples within 2 hours of their collection. If they cannot be immediately tested, they should be stored at 2° C.-8° C., and may be stored for up to 2 days in this manner. After 2 days, they must be stored at -20°(, and may be stored for up to 7 days. Before testing, the sample must recover room temperature, avoid repeated freezing and thawing. It is not recommended that heat inactivated samples be used.

### [Test Method]

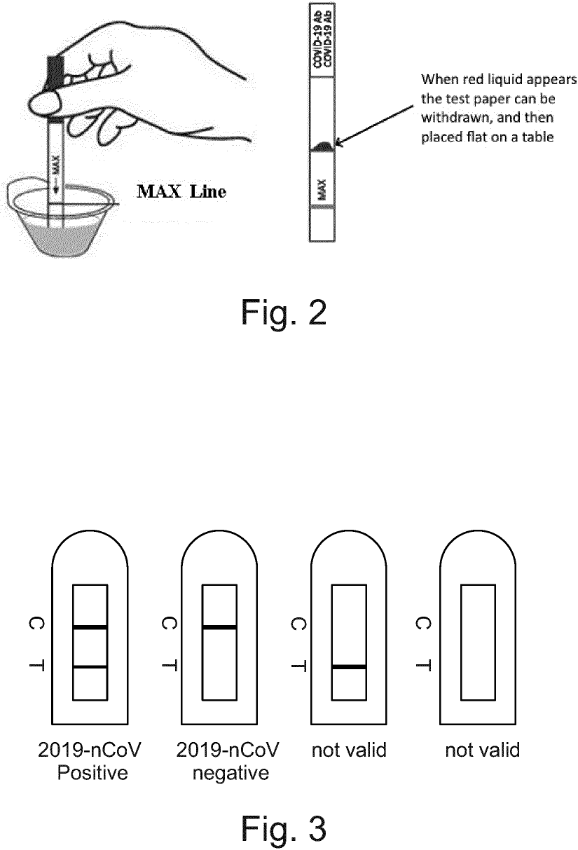

Carefully read the user instructions before testing. The sample for testing, testing reagent and related testing materials must be equilibrated to room temperature, and the test must be conducted under room temperature conditions.
1. Open the tinfoil bag along its margin and then take out the testing strip.
2. Use the urine cup to collect urine, or recover a frozen urine sample to room temperature, and then after placing this into the urine cup, assign it a serial number.
3. Hold the blue end of the testing strip, and insert the side with a MAX line on it into the urine.
WARNING: When inserting the testing stick, the surface of the urine should not go past the MAX line!
4. Wait until a red liquid appears on the white film area, and then take, and then take the testing strip out, and place on a flat table with the film facing upward. Do not place directly in front of a fan or air conditioning for ventilation.
5 . At 10/20 minutes observe the results. Results occurring after 25 minutes have no clinical significance, see FIG. 2. When red liquid appears the test paper can be withdrawn, and then placed flat on a table.

### [Explanation of Testing Results, See FIG. 3]

Not valid: When the quality control area (C) does not have a red line, the test is invalid. It is recommended in this case to use a new test to conduct a new examination, remember when inserting the paper into the urine that this must be done to a sufficient degree.

Positive: There are two red lines, one in the testing area (T) and one in the quality control area (C), with both showing a red reaction line.

Negative: one red line, which is only in the quality control area (C).

### [Limitations of the Testing Method]

1. This reagent is only provided for the testing of human urine samples.
2. The accuracy of this test depends on the sample collection process, if this was done inappropriate, if storage was not appropriate, or if the sample was repeatedly frozen and thawed, then this will influence the test results.
3. The product is only provided in order to carry out qualitative detection of novel coronavirus (2019-nCoV) antibodies in human urine samples, and it cannot accurately measure the number of antibodies the sample. If a test for the amount is needed, then the relevant specialized equipment must be employed.
4. Testing results of this reagent are suppled only for clinical reference, and may not serve as the unitary basis for making a clinical diagnosis, in the clinical management of a patient, they must be taken together with the status of symptoms, disease history, and other laboratory examinations and the efficacy of treatment for comprehensive consideration.
5. Due to the limits of tests using receptor antibody type methods, it is recommended that nucleic acid testing or virus culture identification methods be used in the case of negative results, in order to carry out checking and confirmation.
6. Analysis regarding the possibility of false negative results:
   1) Inappropriate collection of samples, transportation, and processing, and excessively low virus titer in the sample may lead to false negative results.
   2) Genetic alterations in the virus may cause changes in antibody determinates, thereby leading to false negative results.

### [Product Performance Indicators]

1. Positive reference product rate: Industry positive reference product rate must be 5/5.
2. Negative reference product rate: Industry negative reference product rate must be 10/10.
3. Lower testing limit: the industry lower testing limit reference product S1 must be negative, S2 and S3 must be positive.
4. Repeatability: carry out testing with 2 industry repeatable reference products (J1-J2), and these repeatable tests should be done 10 time each, with all of them being positive.
5. Analysis specificity:
   5.1 Cross-reaction: This product does not have a cross reaction with samples that are positive for any of the following: parainfluenza antibodies, influenza virus A antibodies, influenza virus B antibodies, chlamydia pneumoniae antibodies, mycoplasma pneumoniae antibodies, adenovirus antibodies, RSV antibodies, hepatitis B surface antibodies, hepatitis C antibodies, TP-antibodies, HIV antibodies, EB virus antibodies, measles virus antibodies, CMV antibodies, enterovirus 71 antibodies, mumps virus antibodies, the varicella-zoster virus.
   5.2 Interfering substances: the following substances do not influence the testing results of the product: histamine hydrochloride, α-interferon, zanamivir, ribavirin, oseltamivir, peramivir, lopinavir, ritonavir, abidor, levofloxacin, azithromycin, ceftriaxone, meropenem, and tobramycin.
6. Hook effect: The testing results of this product have not displayed any hook effect, within the scope of the titer of clinically positive samples of novel coronavirus.
7. Testing results of this product are not influenced by specific IgM antibodies of destroyed novel coronavirus.

### [Precautions]

1. This item is a one-time use in vitro diagnostic reagent, do not re-use it, and if the item has passed its use-by date, do not use it.
2. Avoid laboratory conditions of excessive ambient temperature, store tests at low temperature, sample diluent must be recovered to room temperature before being opened, in order to avoid moisture absorption.
3. After the test is completed, the testing strip, urine cup, and etc. biological medical waste must be handled in an appropriate manner.
4. There is desiccant within the aluminum foil compound pouch, do not eat it.
5. Do not use repeatedly frozen and thawed sample. During testing, wait until the sample to be tested has equilibrated with room temperature.
6. The sample for testing must be viewed as a potential transmitter of infection, so when processing the sample one must adhere to guidelines for infective disease laboratory operations when carrying out activities. Take heed of biosafety when carrying out activities.
7. Just the same as all other diagnostic reagents, the final diagnosis must be made by a doctor who integrates the testing indications and clinical symptoms, and then makes a determination.

1. A method for detecting infection with or immunity to SARS-Cov-2 of a subject, the method comprising the following steps:
(i) contacting a urine sample from the subject with an application area of a testing device comprising a urine sorption material defining a sequence of said application area, a conjugate area and a testing area, the areas being in direct or indirect capillary flow communication with each other,
(ii) allowing the urine sample to flow by capillarity from said application area to said conjugate area, said conjugate area comprising a testing conjugate movably held therein, wherein said testing conjugate comprises or consists of a polypeptide having an amino acid sequence that shares sequence identity of at least 90%, preferably at least 95%, with a SARS-CoV-2 protein or fragment thereof, coupled to a first colored marker,
(iii) allowing the urine sample to continue to flow by capillarity to the testing area, said testing area comprising a testing sub-area with immobilized anti-human IgA antibodies, and
(iv) determining infection with or immunity to SARS-CoV-2 of the subject by visually inspecting the testing sub-area of the testing area for a color build-up, wherein the presence of a color build-up is indicative of said infection with or immunity to SARS-CoV-2.

2. The method as claimed in claim 1, wherein the SARS-CoV-2 protein or fragment thereof has an amino acid sequence comprising that of SARS-CoV-2 surface glycoprotein (Coronavirus S protein), of SARS-CoV-2 surface glycoprotein S1 (Coronavirus S1 protein), of SARS-CoV-2 surface glycoprotein S2 (Coronavirus S2 protein), of SARS-CoV-2 RBD protein, of SARS-CoV-2 Nucleocapsid phosphoprotein N (Coronavirus N protein), or of fragments of at least 150 consecutive amino acids thereof.

3. The method as claimed in claim 1 wherein the polypeptide has an amino acid sequence that shares sequence identity of at least 90%, preferably at least 95%, with SEQ ID NO:2, SEQ ID NO:3, SEQ ID NO:4, SEQ ID NO:5, SEQ ID NO:6, SEQ ID NO:7, SEQ ID NO:8, or any fragment of at least 150 consecutive amino acids thereof.

4. The method as claimed in claim 1 wherein said first colored marker is selected among nanometer-sized gold particles and latex particles.

5. The method as claimed in claim 1 wherein said conjugate area further comprises a control conjugate movably held therein, wherein said control conjugate comprises a control polypeptide coupled to a second colored marker and said testing area comprising a control sub-area with immobilized anti-(control polypeptide) antibodies, and wherein the method further comprises, before or after step (iv), the step of
(iv′) determining a correct sample flow within the testing device by visually inspecting the control sub-area of the testing area for a color build-up, wherein the presence of a color build-up is indicative of said correct sample flow within the testing device.

6. The method as claimed in claim 5, wherein the control polypeptide is selected among immunoglobulins, preferably IgA, IgG, more preferably non-human IgG, such as rabbit IgG or mouse IgG.

7. The method as claimed in claim 5 wherein said second colored marker is selected among nanometer-sized or colloid gold particles or latex particles.

8. The method as claimed in claim 5 wherein the absence of color build-up in the control sub-area in step (iv′) is indicative that the test result of step (iv) should be disregarded and the method repeated with a fresh testing device.

9. The method as claimed in claim 1, wherein the urine sorption material of the testing device is supported on a backing pad, preferably made of glass fiber, polyester film or a non-woven.

10. A testing device for detecting infection with or immunity to SARS-Cov-2 of a subject, the testing device comprising a urine sorption material defining a sequence of an application area, a conjugate area and a testing area, the areas being in direct or indirect capillary flow communication with each other, wherein said application area is configured for receiving a urine sample of a subject, wherein said conjugate area comprises a testing conjugate movably held therein, wherein said testing conjugate comprises or consists of a polypeptide having an amino acid sequence that shares sequence identity of at least 90%, preferably at least 95%, with a SARS-CoV-2 protein or fragment thereof, coupled to a first colored marker, wherein said testing area comprises a testing sub-area with immobilized anti-human IgA antibodies, and wherein said testing sub-area is configured for visually inspecting any color build-up, and wherein a color build-up is indicative of said infection with or immunity to SARS-CoV-2 of the subject.

11. The testing device as claimed in claim 10, wherein the SARS-CoV-2 protein or fragment thereof has an amino acid sequence comprising that of SARS-CoV-2 surface glycoprotein (Coronavirus S protein), of SARS-CoV-2 surface glycoprotein S1 (Coronavirus S1 protein), of SARS-CoV-2 surface glycoprotein S2 (Coronavirus S2 protein), of SARS-CoV-2 RBD protein, of SARS-CoV-2 Nucleocapsid phosphoprotein N (Coronavirus N protein), or of fragments of at least 150 consecutive amino acids thereof.

12. The testing device as claimed in claim 10 wherein the polypeptide has an amino acid sequence that shares sequence identity of at least 90%, preferably at least 95%, with SEQ ID NO:2, SEQ ID NO:3, SEQ ID NO:4, SEQ ID NO:5, SEQ ID NO:6, SEQ ID NO:7, SEQ ID NO:8, or any fragment of at least 150 consecutive amino acids thereof.

13. The testing device as claimed in claim 10 wherein said first colored marker is selected among nanometer-sized gold particles and latex particles.

14. The testing device as claimed in claim 10 wherein said conjugate area further comprises a control conjugate movably held therein, wherein said control conjugate comprises a control polypeptide coupled to a second colored marker and said testing area comprising a control sub-area with immobilized anti-(control polypeptide) antibodies upstream or downstream of the testing sub-area, and wherein said control sub-area is configured visually inspecting any color build-up, wherein a color build-up is indicative of a correct sample flow within the testing device.

15. The testing device as claimed in claim 14, wherein the control polypeptide is selected among immunoglobulins, preferably IgA, IgG, more preferably non-human IgG, such as rabbit IgG or mouse IgG.

16. The testing device as claimed in claim 14 wherein said second colored marker is selected among nanometer-sized or colloid gold particles and latex particles.

17. The testing device as claimed in claim 10 wherein the urine sorption material is supported on a backing pad, preferably made of glass fiber, polyester film or a non-woven.

18. Use of a testing conjugate comprising or consisting of a polypeptide having an amino acid sequence that shares sequence identity of at least 90%, preferably at least 95%, with a SARS-CoV-2 protein or fragment thereof, coupled to a first colored marker, for detecting infection/immunity of a subject with SARS-Cov-2, wherein detecting infection with or immunity to SARS-Cov-2 of a subject is made based on the presence of SARS-CoV-2 specific IgA and/or IgG antibodies in a urine sample of said subject.

19. Use as claimed in claim 18, wherein the SARS-CoV-2 protein or fragment thereof is an amino acid sequence comprising that of SARS-CoV-2 surface glycoprotein (Coronavirus S protein), of SARS-CoV-2 surface glycoprotein S1 (Coronavirus S1 protein), of SARS-CoV-2 surface glycoprotein S2 (Coronavirus S2 protein), of SARS-CoV-2 RBD protein, of SARS-CoV-2 Nucleocapsid phosphoprotein N (Coronavirus N protein), or of fragments of at least 150 consecutive amino acids thereof, the polypeptide preferably having an amino acid sequence that shares sequence identity of at least 90%, still more preferably at least 95%, with SEQ ID NO:2, SEQ ID NO:3, SEQ ID NO:4, SEQ ID NO:5, SEQ ID NO:6, SEQ ID NO:7, SEQ ID NO:8, or any fragment of at least 150 consecutive amino acids thereof.


SEQUENCE LISTING (8 sequences)

SEQ ID NO: 1 - DNA, 3840 bp, SARS-CoV-2
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacccatcatcaccatcaccattga

SEQ ID NO: 2 - protein, 1279 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTHHHHHH

SEQ ID NO: 3 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 608 aa, SARS-CoV-2
DIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKL

SEQ ID NO: 5 - protein, 194 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 6 - protein, 670 aa, SARS-CoV-2
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 7 - protein, 528 aa, SARS-CoV-2
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 8 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA